(19) United States
(12) Patent Application Publication
(10) Pub. No.: US 2015/0057747 A1
(43) Pub. Date: Feb. 26, 2015

(54) STENT WITH ALTERNATIVE CELL SHAPES

(71) Applicant: St. Jude Medical, Cardiology Division, Inc., St. Paul, MN (US)

(72) Inventors: Xue Mei Li, Shoreview, MN (US); Yousef F. Alkhatib, Edina, MN (US)

(21) Appl. No.: 14/464,834

(22) Filed: Aug. 21, 2014

Related U.S. Application Data
(60) Provisional application No. 61/868,657, filed on Aug. 22, 2013.

(51) Int. Cl. A61F 2/24; A61F 2/90

(52) U.S. Cl. CPC A61F 2/2418; A61F 2/90; A61F 2230/0026
USPC 623/2.18

(57) ABSTRACT

A stent for use with a prosthetic heart valve for replacement of a native valve includes an expandable stent body having a collapsed configuration and an expanded configuration, with an annulus section at an inflow end of the stent body and an aortic section at an outflow end. A plurality of commissure attachment features are positioned circumferentially around the stent body. A distal edge of the annulus section is generally parabolic and extends from one of the commissure attachment features to an adjacent one of the commissure attachment features, with the middle of the parabola being closest to the inflow end of the stent body.

### CROSS-REFERENCE TO RELATED APPLICATIONS

The present application claims the benefit of the filing date of U.S. Provisional Patent Application No. 61/868,657 titled “STENT WITH ALTERNATIVE CELL SHAPES,” filed Aug. 22, 2013, the disclosure of which is hereby incorporated by reference herein.

### BACKGROUND OF THE INVENTION

Certain prosthetic heart valves incorporate an expandable stent body and valve elements such as prosthetic valve leaflets mounted to the stent body. Valves of this type may be implanted in the heart by advancing the valve into the body of a patient with the stent body in a collapsed or crimped condition in which the stent body has a relatively small diameter. Once the valve is positioned at the desired implantation site, the stent body is brought to an expanded condition in which the stent body bears on the surrounding native tissue and holds the valve in place. The valve acts as a functional replacement for a diseased native valve. Thus, the valve elements inside the stent body permit blood flow in the antegrade direction but substantially block flow in the opposite, retrograde direction. For example, a prosthetic valve may be advanced to a site within a diseased native aortic valve percutaneously through the arterial system and into the aorta to the native aortic valve. In a transapical placement, a prosthetic valve may be advanced through an incision in the apex of the heart and through the left ventricle to the native aortic valve. Other approaches through other access sites can be used. Once the prosthetic valve is in place, it permits flow from the left ventricle into the aorta when the left ventricle contracts during systole, but substantially blocks retrograde flow from the aorta into the left ventricle during diastole.

There are significant challenges in the design of an expandable stent body and valve. For example, the stent body desirably can be collapsed to a relatively small diameter to facilitate advancement into the body, such as through a catheter or other introducer. However, the stent body must be capable of expanding to an operative, expanded condition in which the stent body securely engages the surrounding native tissues to hold the valve in place.

Terms including retrieval, resheathing, repositioning, and removal refer to the ability to return the prosthetic valve to the delivery apparatus after it has been wholly or partly deployed from the apparatus. Such retrieval may be done to allow the valve to be moved to another, different location or orientation in the patient (so-called repositioning), or to completely remove the valve from the patient (so-called removal). Returning the valve to the delivery apparatus involves re-collapsing the valve to its reduced circumferential size. If the valve is to be repositioned, then the delivery apparatus is moved until it reaches the desired new location or orientation in the patient. The valve can then be advanced out of the delivery apparatus again, allowing it to again expand (or be expanded) to its full operating size.

FIG. 1 shows an example of one type of prior art collapsible prosthetic heart valve. Examples of collapsible prosthetic heart valves are described in U.S. Application Publication No. 2012/0053681 and U.S. Design Patent No. D660,967, the entire contents of which are hereby incorporated by reference herein. In its fully-expanded, unconstrained configuration, an expandable stent body 10 may include an annulus section 12, an aortic section 20, and a coronary section 15 between the annulus section and the aortic section. The annulus section 12 in the expanded configuration may be generally in the form of a cylindrical tube having a central axis 14, whereas aortic section 20 may be generally in the form of a hoop coaxial with the annulus section. For purposes of clarity of description, stent body 10 may be thought of as comprising a plurality of struts, even in the case in which the stent body is formed from a unitary structure. For example, annulus section 12 may be defined, at least in part, by a plurality of annulus struts 16. Similarly, aortic section 20 may be defined, at least in part, by a plurality of aortic struts 26. Coronary section 15 may be defined, at least in part, by portions of annulus struts 16 and aortic struts 26. As used herein, a strut is a portion of a cell of stent body 10.

Annulus section 12 includes numerous cells defined by annulus struts 16 which join one another at intersection points. These cells are disposed in a proximal row 70 and a distal row 60, each such row extending circumferentially around the proximal-to-distal axis 14 so that the cells cooperatively form a generally cylindrical wall. Annulus section 12 may have a single row of cells or more than two rows of cells depending on the size of the cells. In the expanded condition, the annulus struts 16 of each cell form a generally diamond-shaped structure. In the unexpanded, crimped or collapsed configuration, the annulus struts 16 of each cell extend substantially in the proximal and distal directions, so that each cell is collapsed in the circumferential direction.

In the prosthetic valve shown in FIG. 1, the valve leaflets 50 are attached to a cuff 55 and commissure attachment features 40 and are positioned inside the stent body 10 so as to overlap portions of annulus struts 16 and/or aortic struts 26 in, for example, overlap area 90. The cuff 55 may be attached to the stent body 10, for example, by sutures 57. This configuration results in a relatively large profile for the prosthetic valve when in the collapsed configuration. A relatively large profile for the prosthetic valve in the collapsed configuration may be undesirable, for example, because a larger delivery device may be required to accommodate such a prosthetic heart valve. Overlapping of struts 16, 26 and leaflets 50 may also be undesirable because, for example, the leaflets may be more likely to be damaged when the prosthetic heart valve is in the collapsed configuration.

The size of the profile of a prosthetic heart valve in the collapsed configuration is just one consideration involved in designing an expandable prosthetic heart valve. For example, the prosthetic heart valve desirably can collapse to a relatively small diameter without damaging the flexible leaflets of the valve, and preferably can be easily re-collapsed and resheathed (e.g., for repositioning) after expansion or partial expansion at the implant site in the patient.

### BRIEF SUMMARY OF THE INVENTION

Features described herein provide for a prosthetic heart valve having a partially discontinuous cellular stent structure that may collapse to a smaller profile than, for example, heart valves having stents with continuous cellular structures. This structure may be achieved, for example, by removing a number of struts from the annulus and/or coronary section of the stent, thus reducing the amount of material in the stent body. To prevent the appearance of catch points (which can make resheathing the valve during delivery difficult or impossible), struts forming a distal edge of the annulus section may have a generally parabolic or a general “U” or “V” shape. Thus, catch points may be avoided while still achieving a reduction in stent material.

In one embodiment, a stent for use with a prosthetic heart valve for replacement of a native valve includes an expandable stent body having a collapsed configuration, an expanded configuration, an inflow end, an outflow end, and an annulus section at the inflow end. The stent may include at least one circumferential row of cells defined by struts of the stent body. The at least one row of cells may include a first cell, a second cell, and a third cell. The second cell may be positioned between the first and third cells. The annulus section may include a distal edge defined by a series of at least three struts, a first one of the struts defining a portion of the first cell, a second one of the struts defining a portion of the second cell, and a third one of the struts defining a portion of the third cell. Each point on the second one of the struts may be nearer, or equally near, the inflow end compared to each point on the first one of the struts and the third one of the struts.

In another embodiment, a stent for use with a prosthetic heart valve for replacement of a native valve may include an expandable stent body having a collapsed configuration and an expanded configuration. The stent may further include an annulus section at an inflow end of the stent body, an aortic section at an outflow end of the stent body, and a plurality of commissure attachment features positioned circumferentially around the stent body. A distal edge of the annulus section may be generally parabolic and may extend from one of the commissure attachment features to another of the commissure attachment features.

### BRIEF DESCRIPTION OF THE DRAWINGS

FIG. 1 is a perspective view of a prior art prosthetic heart valve in an expanded configuration.

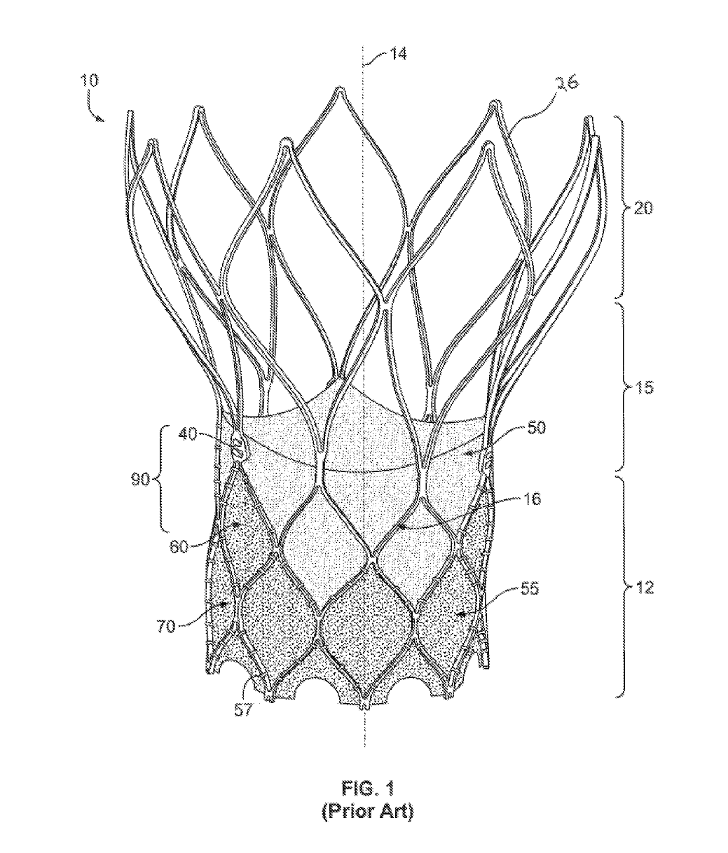

FIG. 2 is a perspective view of a stent body according to an aspect of the invention.

FIG. 3 is perspective partial view of the stent body of FIG. 2 with a prosthetic heart valve leaflet in an expanded configuration, with two thirds of the prosthetic heart valve omitted for clarity of illustration.

FIGS. 4A-D are partial views of examples of truncated diamond cell shapes in a stent body according to aspects of the invention.

### DETAILED DESCRIPTION

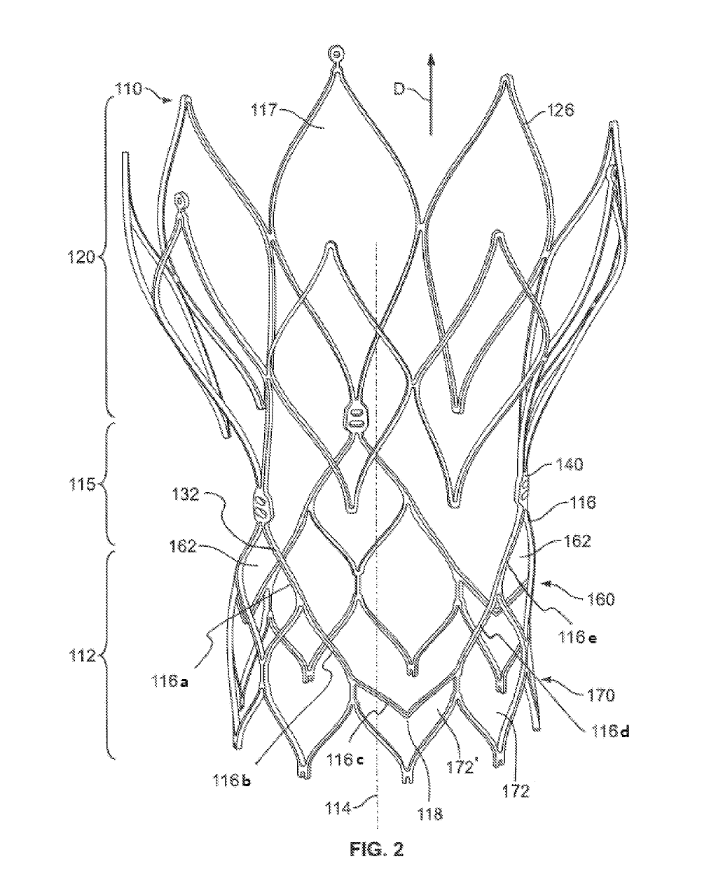

FIG. 2 illustrates an expandable stent body 110 according to an embodiment of the disclosure. Stent body 110 is illustrated without prosthetic valve leaflets attached for the sake of clarity. The expandable stent body 110 may be formed as a unitary structure, for example, by laser cutting or etching a tube of a superelastic metal alloy such as a nickel-titanium alloy, which can be of the type sold under the designation Nitinol. Such a unitary structure can also be referred to as a “non-woven” structure, in that it is not formed by weaving or winding one or more filaments. In its fully-expanded, unconstrained configuration, the illustrated stent body 110 includes an annulus section 112, an aortic section 120, and a coronary section 115 between the annulus section and the aortic section. Stent body 110 includes an annulus section 112 near an inflow end of the stent body, an aortic section 120 near an outflow end of the stent body, and a coronary section 115 between the annulus and aortic sections 112 and 120. Annulus section 112 may be defined, at least in part, by a plurality of annulus struts 116. Similarly, aortic section 120 may be defined, at least in part, by a plurality of aortic struts 126. Coronary section 115 may be defined, at least in part, by portions of annulus struts 116 and aortic struts 126. A portion of annulus section 112 in the expanded configuration is generally in the form of a cylindrical, truncated-conical, or revolved parabolic tube having a central axis 114, whereas aortic section 120 is generally in the form of a hoop coaxial with the annulus section. Annulus section 112 can have other shapes, such as elliptical or triangular, depending on the patient's anatomy. Not all embodiments of stent body 110 include an aortic section 120. For example, stents for aortic valves, as well as stents for bicuspid and/or mitral valves, may not include an aorta section 120.

Stent body 110 is adapted for implantation in the body of a patient with annulus section 112 within the native aortic valve annulus and with aortic section 120 within the sinotubular junction and/or aorta. In this configuration, blood flows in through annulus section 112 and out through aortic section 120. Further, in this embodiment, annulus section 112 of stent body 110 may be configured to be the leading end during delivery. In other words, annulus section 112 may be configured to be advanced out of a delivery device prior to aortic section 120 advancing out of the delivery device during delivery of the stent body. Thus, when the valve incorporating stent body 110 is placed in the patient, aortic section 120 will be disposed distal to annulus section 112 in the frame of reference of the patient's circulatory flow. Accordingly, as used with reference to features of the stent body and valve, the direction D along axis 114 from annulus section 112 towards aortic section 120 is referred to as the distal direction, and the opposite direction is taken as the proximal direction. Stated another way, the “distal direction” along the stent body is the direction of antegrade blood flow when the stent body is properly implanted in the native aortic annulus. Directions toward and away from axis 114 are also referred to herein as “radial directions.” As used with reference to features of the stent body, “circumferential directions” are the directions around axis 114.

Annulus section 112 includes a set of commissure features 140 formed integrally with the remainder of the stent body. Commissure features 140 are found at three locations spaced equally around the circumference of the annulus section. Commissure features 140, aortic struts 126 extending distally therefrom and annulus struts 116 extending proximally therefrom, may serve as the only connection between annulus section 112 and aortic section 120. Each commissure feature 140 may include one or more eyelets therein. Three prosthetic valve leaflets (not illustrated in FIG. 2) may be attached to commissure features 140, such as by suturing through the eyelets thereof, so that the leaflets are disposed within annulus section 112 of the stent body. A lining or “cuff” (not illustrated in FIG. 2) may be provided on the interior surface, exterior surface or both surfaces of annulus section 112, over all or part of the axial extent of the annulus section. The leaflets and cuff may be formed from conventional biocompatible materials such as synthetic polymers and animal tissues such as pericardial tissues. Stent body 110 can include more than three or fewer than three commissure features 140, for example, to accommodate more or fewer than three valve leaflets.

Stent body 110 is formed with a relatively open coronary section 115. For example, in one embodiment, the distal edge 132 of annulus section 112 may have one or more generally parabolic sections, or may generally take the form of one or more catenary or “U”-shaped or “V”-shaped sections. As illustrated in FIG. 2, the distal edge 132 of annulus section 112 may be defined, at least in part, by a series of connecting annulus struts 116a-e. The distal edge 132 of annulus section 112 may extend continuously along the series of connecting annulus struts 116a-e so as to span two or more cells located on an opposite side of annulus struts 116a-e.

As shown in FIG. 2, such cells can include annulus cells 172, 172′, which are positioned on a proximal side of annulus struts 116a-e opposite distal edge 132 which, in part, provides for open coronary section 115. In an example, the series of annulus connecting struts 116a-e have a proximalmost point or section 118 near the center of annulus strut 116c with distalmost portions near the ends of annulus struts 116a, 116e which may connect with commissure features 140. Stated another way, the proximalmost point or section 118 is relatively close to the inflow end of stent body 110, and the distalmost portions are more distant from the inflow end than the proximalmost point or section. Although there is stent structure proximal to proximalmost point or section 118, proximalmost point 118 is the most proximal point on the series of annulus connecting struts 116a-e. It should be understood that, although described herein as a series of connecting annulus struts 116a-e, each of the connecting annulus struts 116a-e may form part of a unitary stent body 110. It should further be understood that the distal edge 132 of annulus section 112 may be defined by more or fewer struts, such as annulus connecting struts 116b-d. Thus, the distal edge 132 of annulus section 112 116 need not actually connect to commissure features 140, and could connect to points of stent body 110 other than the commissure features.

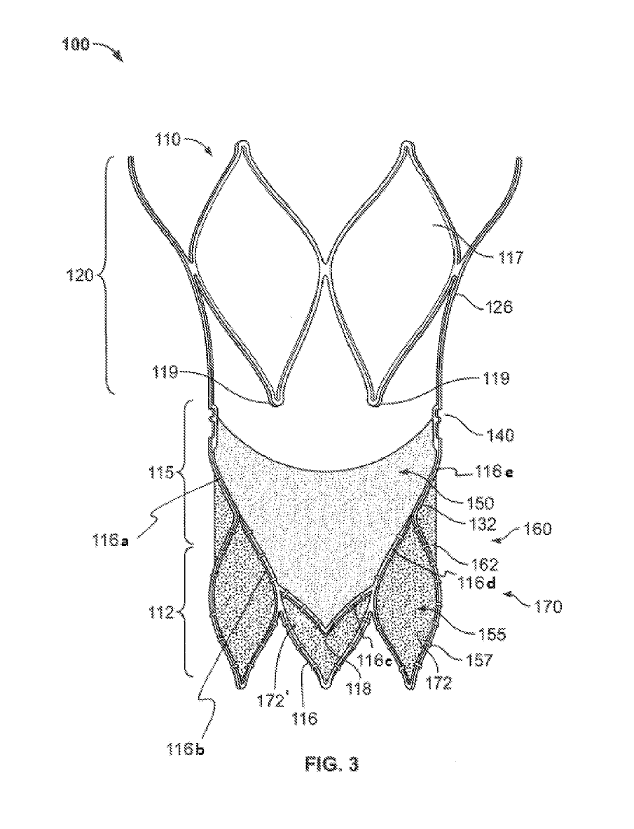

It should further be understood that the term “generally parabolic” is not limited to the strict mathematical definition of a parabola. Rather, the distal edge 132 of annulus section 112 has a proximalmost point or section 118. Moving in either circumferential direction from the proximalmost point or section, each further point on the distal edge 132 of annulus section 112 is positioned at the same distance from the proximal end of the stent body or distal to the immediately preceding point. In other words, if the distal edge 132 of annulus section 112 is defined by a series of connecting annulus struts 116a-e, the connecting struts are shaped to have a proximalmost point or section 118 (or minimum). In FIG. 3, this point is illustrated near the center of the series of annulus connecting struts 116a-e. Moving in the circumferential direction from that minimum point 118 to, for example, a commissure attachment feature 140, each portion of each strut 116a-e in the series is positioned at the same distance from the proximal end of the stent body or distal to the immediately preceding point. Thus, both a “U” shape and a “V” shape would be considered “generally parabolic.” In fact, a “V” shape may be preferred to facilitate crimping or collapsing the stent body 110, but again, both “U”-shapes and “V”-shapes are included in the term “generally parabolic.”

FIG. 3 illustrates a prosthetic heart valve 100, including stent body 110 and prosthetic valve leaflets 150, according to an embodiment of the disclosure. In FIG. 3, only a portion of the full heart valve 100 is illustrated, with two thirds of the heart valve omitted for clarity of illustration. In other words, prosthetic heart valve 100 is shown with one of three leaflets 150, and the portion of stent body 110 corresponding to the one leaflet. As described above, a cuff 155 may be attached to stent body 110. A distal end of the cuff 155 may be attached to stent body 110 along the distal edge 132 of annulus section 112, for example by sutures 157 that follow the series of annulus connecting struts 116a-e. The cuff 155 may not extend distal of the distal edge 132 of annulus section 112.

In the configuration described above, annulus section 112 can include both a proximal row 170 of cells 172 that extends circumferentially around stent body 110 and a distal row 160 of cells 162. The shape of the distal edge 132 of annulus section 112 results in some cells 172 in proximal row 170 having different shapes than other cells 172′. For example, some cells 172 in proximal row 170 may have a generally diamond or parallelogram shape represented by two opposing pairs of generally parallel annulus struts 116. One of the annulus struts 116 may comprise a portion of the distal edge 132 of annulus section 112 and/or one of the series of annulus connecting struts 116a-e. For example, in the illustrated embodiment, annulus strut 116b is one of two opposing annulus struts 116 that form part of a cell, while it also is one of the series of annulus connecting struts and defines a portion of the distal edge 132 of annulus section 112. Other cells 172′ in proximal row 170 may take partial, half, or truncated diamond or parallelogram shapes. In the illustrated embodiment, proximal row 170 includes a truncated diamond-shaped cell 172′ near the proximalmost or minimum point 118 of the distal edge 132 of annulus section 112, the truncated diamond-shaped cell being adjacent full diamond-shaped cells 172 on either side thereof.

In one example, a truncated diamond-shaped cell 172′ is positioned between full diamond-shaped cells 172, and annulus connecting struts 116b-d each define portions of the truncated diamond-shaped cell or the full diamond-shaped cells. Thus, in this particular example, a portion of the truncated diamond-shaped cell is defined by annulus connecting strut 116c, and portions of the adjacent full diamond-shaped cells are defined by annulus connecting struts 116b, 116d. The annulus connecting struts 116b-d define, at least in part, the distal edge 132 of annulus section 112. In this example, no point on the annulus connecting struts 116b, d of the distal edge 132 is closer to the inflow end of stent body 110 than any point on annulus connecting strut 116c. Stated another way, each point on the distal edge 132 of annulus section 112 that defines a portion of the truncated diamond-shaped cell 172′ is nearer, or equally near, the inflow end of stent body 110 compared to each point on the distal edge of the annulus section defining portions of the two full diamond-shaped cells 172.

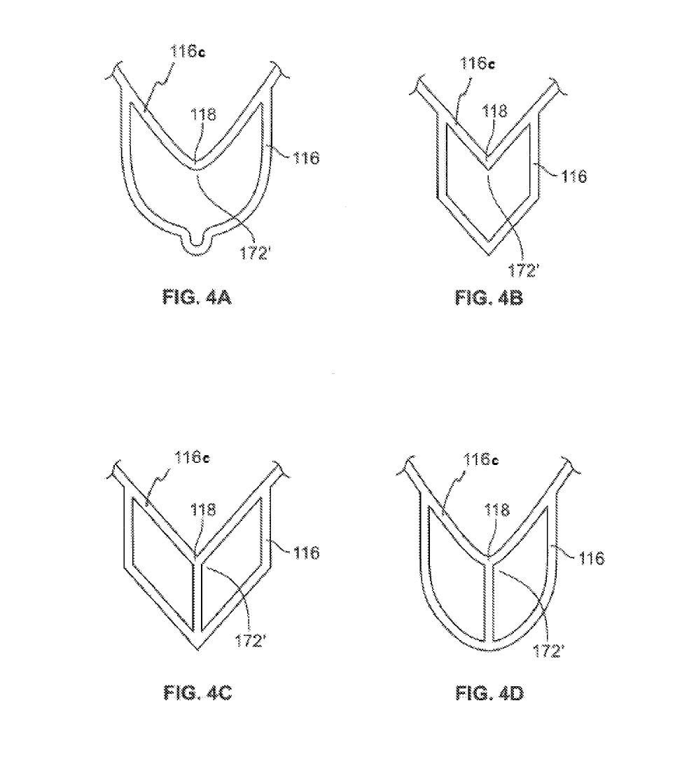

The description of a shape as a truncated diamond can encompass a number of different specific forms. FIGS. 4A-D illustrate various example shapes of cells 172′ that may be considered a truncated diamond shape. In these examples, each truncated diamond shape includes a proximalmost point or section 118, comprising a portion of annulus connecting strut 116c, which defines a portion of the distal edge 132 of annulus section 112. More particularly, FIG. 4A illustrates a cell 172′ with the general shape of a truncated diamond including generally curved distal and proximal sections. FIG. 4B illustrates a cell 172′ with the general shape of a truncated diamond including two substantially straight distal sections and two substantially straight proximal sections. FIGS. 4C-4D illustrate cells 172′ with the general shapes of truncated diamonds generally each resembling a pair of parallelogram shapes. Cell 172′ in FIG. 4C includes two substantially straight distal sections and two substantially straight proximal sections connected by a vertical strut. Cell 172′ in FIG. 4D includes two curved distal sections and two curved proximal sections connected by a vertical strut. Other possible forms could be considered truncated diamonds as well. Such forms can include various combinations of the features shown in the examples or can be derived based on the general principles of the examples shown and described herein.

The alternating pattern of diamond and truncated diamond cell shapes could, for example, be repeated for each leaflet 150 in the prosthetic heart valve 100. In a prosthetic heart valve 100 with three leaflets 150, the pattern of three cells described above could be repeated an additional two times around stent body 110, one time for each additional leaflet. Although the distal edge 132 of annulus section 112 of stent body 110 has been described as including a portion, such as the series of annulus connecting struts 116a-e, extending between commissure features 140 and defining, at least in part, a group of two generally diamond-shaped cells 172 bordering a truncated diamond-shaped cell 172′, other configurations are possible. For example, the distal edge 132 of annulus section 112 may span more than three cells, with the cells nearest the proximalmost point or section 118 of the distal edge 132 of annulus section 112 having a diamond shape with the most truncation, and the cells relatively farther away from the proximalmost point or section having less truncated diamond shapes or full diamond shapes.

Distal row 160 of cells 162 may be significantly interrupted by the open coronary section 115 defined, at least in part, by the distal edge 132 of annulus section 112. The coronary section 115 may also be defined, at least in part, by a continuous circumferential row of cells in aortic section 120. In the embodiment illustrated in FIGS. 2-3, distal row 160 of cells 162 includes generally diamond- or parallelogram-shaped cells only under commissure features 140 and bounded by portions of the distal edge 132 of annulus section n112. Cells that would otherwise exist in distal row 160 between commissure features 140 in the circumferential direction are completely omitted in the illustrated embodiment. These gaps, present by omission of at least some of the structure of stent body 110 that otherwise would define such cells, results in less material in the stent body, which helps to reduce the profile of the prosthetic heart valve 100 in the collapsed or crimped configuration. In other words, open coronary section 115 has fewer cells than other stent bodies. In the illustrated embodiment, open coronary section 115 has fewer cells than each of annulus section 112 and aortic section 120. Preferably, the portion of stent body 110 omitted corresponds to an area in which annulus and/or aortic struts 116, 126, a portion of a cuff, and a leaflet 150 are traditionally positioned, resulting in only the leaflet adding to the profile of prosthetic heart valve 100 at that location, rather than a combination of struts, cuff, and leaflets adding to the profile of the prosthetic heart valve.

A reduction in crimping profile is possible by removing portions of cells or complete cells only in distal row 160. However, if no additional modifications were made, the proximal row of cells 170 would remain unaltered, with at least some cells taking the form of diamond or parallelogram shapes with free distal ends, i.e., distal ends that do not connect to any other cells. The free distal ends of these cells would create catch points in stent body 110, such that resheathing could be difficult or impossible. For example, during delivery of prosthetic heart valve 100, stent body 110 is loaded in a delivery device (not illustrated) in a crimped or collapsed configuration prior to being positioned in the heart. Once near the native heart valve, annulus section 112 of stent body 110 is released from the delivery device and begins to expand prior to aortic section 120 being released from the delivery device. Even if the entire proximal row of cells 170 exits the delivery device and begins expanding, the entire stent body 110 may still be resheathed into the delivery device if the initial positioning is undesirable. If cell 172′ had the shape of a full diamond or parallelogram with a free distal end, that free distal end could easily catch on the delivery device during resheathing. By providing cells 172′ with partial or truncated diamond or parallelogram shapes, free distal ends, and thus possible catch points, are eliminated from the design of proximal row 170 of stent body 110.

It should be noted that aortic section 120 may include a number of cells 117 forming generally diamond or parallelogram shapes with free proximal ends 119. Free proximal ends 119 of cells 117 do not create a risk of catching during resheathing as long as annulus section 112 of stent body 110 is the leading end during delivery of prosthetic heart valve 100.

The configuration of open coronary section 115 may have benefits in addition to providing a reduced crimping profile of prosthetic heart valve 100, while simultaneously retaining resheathability. For example, the reduced material of stent body 110 in the coronary section 115 may provide for better coronary perfusion when prosthetic heart valve 100 is implanted. Further, the configuration of the distal edge 132 of annulus section 112 provides an attachment site for the lower belly of leaflet 150. The expanded diameter of annulus section 112 may be larger than that of coronary section 115 to provide additional anchoring force. The shape of annulus section 112 could be, for example, generally cylindrical, generally conical, or generally revolved parabolic. Coronary section 115 could be, for example, generally cylindrically shaped or generally spherical or ball shaped.

In some embodiments, aortic section 120 may be optional. Generally, aortic section 120 provides valve alignment and reinforcement to coronary section 115. However, if the combination of coronary section 115 and annulus section 112 provides enough functional force, aortic section 120 may be omitted.

As described previously, FIG. 3 illustrates a third of prosthetic heart valve 100. As such, the complete prosthetic heart valve 100 may include additional instances of elements described above. For example, prosthetic heart valve 100 may include two additional prosthetic valve leaflets 150 with corresponding additional cells 172, 172′, 162 in a full stent body 110 (as shown in FIG. 2), as well as additional corresponding struts. Further, although described generally in relation to a prosthetic tricuspid valve, embodiments of the prosthetic heart valve may be altered to include more or fewer valve leaflets, with more or fewer corresponding structures to accommodate the valve leaflets.

Although the current disclosure includes a description of particular embodiments, it is to be understood that these embodiments are merely illustrative of the principles and applications of the present disclosure. It is therefore to be understood that numerous modifications may be made to the illustrative embodiments, including combining elements of different embodiments, and that other arrangements may be devised without departing from the spirit and scope of the present invention as defined by the appended claims.

It will be appreciated that the various dependent claims and the features set forth therein can be combined in different ways than presented in the initial claims. It will also be appreciated that the features described in connection with individual embodiments may be shared with others of the described embodiments.

The following Paragraphs summarize certain aspects of the disclosure.

Paragraph A: A stent for use with a prosthetic heart valve for replacement of a native valve, the stent comprising: an expandable stent body having a collapsed configuration, an expanded configuration, an inflow end, an outflow end, and an annulus section at the inflow end; at least one circumferential row of cells defined by struts of the stent body, the at least one row of cells including a first cell, a second cell, and a third cell, the second cell being positioned between the first and third cells; wherein a distal edge of the annulus section is defined by a series of at least three struts, a first one of the struts defining a portion of the first cell, a second one of the struts defining a portion of the second cell, and a third one of the struts defining a portion of the third cell; wherein each point on the second one of the struts is nearer, or equally near, the inflow end compared to each point on the first one of the struts and third one of the struts.

Paragraph B: The stent of Paragraph A, wherein the second cell is directly adjacent each of the first and third cells.

Paragraph C: The stent of Paragraph A, wherein the stent body includes an aortic section at the outflow end, and a coronary section between the annulus section and the aortic section.

Paragraph D: The stent of Paragraph C, wherein the distal edge of the annulus section includes a generally parabolic section that extends across at least part of a circumference of the stent body, the generally parabolic section having one proximal section that is relatively close to the inflow end and at least two distal sections that are more distant from the inflow end than the one proximal section.

Paragraph E: The stent of Paragraph D, wherein the one proximal section of the generally parabolic section defines at least part of the second cell.

Paragraph F: The stent of Paragraph E, wherein the annulus section includes at least one continuous circumferential row of cells, the aortic section includes at least one continuous circumferential row of cells, and the coronary section includes fewer cells than each of the annulus section and the aortic section.

Paragraph G: The stent of Paragraph F, wherein the coronary section is at least partially defined by the at least one continuous circumferential row of cells in the aortic section and the distal edge of the annulus section.

Paragraph H: The stent of Paragraph E, wherein the generally parabolic section of the annulus section is defined by at least five connecting struts extending at least partially around the circumference of the stent body.

Paragraph I: The stent of Paragraph D, further comprising a plurality of commissure attachment features.

Paragraph J: The stent of Paragraph I, wherein one of the distal sections is connected to a first one of the commissure attachment features and the other of the distal sections is connected to a second one of the commissure attachment features.

Paragraph K: A prosthetic heart valve, comprising the stent of Paragraph D and a plurality of prosthetic valve leaflets mounted inside the stent body.

Paragraph L: The prosthetic heart valve of Paragraph K, wherein at least one of the prosthetic valve leaflets is attached to the stent body along a portion of the distal edge of the annulus section.

Paragraph M: A stent for use with a prosthetic heart valve for replacement of a native valve, the stent comprising: an expandable stent body having a collapsed configuration and an expanded configuration; an annulus section at an inflow end of the stent body; an aortic section at an outflow end of the stent body; and a plurality of commissure attachment features positioned circumferentially around the stent body, wherein a distal edge of the annulus section is generally parabolic and extends from one of the commissure attachment features to another of the commissure attachment features.

Paragraph N: The stent of Paragraph M, wherein the distal edge of the annulus section is defined by a plurality of struts extending at least partially around the circumference of the stent body.

Paragraph O: The stent of Paragraph M, wherein the distal edge of the annulus section is defined by at least three connecting struts extending at least partially around the circumference of the stent body.

Paragraph P: The stent of Paragraph O, wherein the at least three connecting struts each define at least a portion of each of a plurality of adjacent cells in the annulus section.

Paragraph Q: The stent of Paragraph P, wherein, when the stent body is in the expanded configuration, a first one of the cells is generally diamond-shaped.

Paragraph R: The stent of Paragraph Q, wherein a second one of the cells includes a curved distal section and a curved proximal section.

Paragraph S: The stent of Paragraph Q, wherein a second one of the cells includes at least two substantially straight distal sections and at least two substantially straight proximal sections.

Paragraph T: The stent of Paragraph S, wherein the at least two substantially straight distal sections are connected to the at least two substantially straight proximal sections by a vertical strut.

Paragraph U: The stent of Paragraph Q, wherein a second one of the cells includes at least two curved distal sections connected to at least two curved proximal sections by a vertical strut.

Paragraph V: The stent of Paragraph Q, wherein the distal edge of the annulus section has one proximal section that is relatively close to the inflow end and at least two distal sections that are more distant from the inflow end than the one proximal section, the one proximal section defining a portion of a second one of the cells.

Paragraph W: The stent of Paragraph V, wherein a third one of the cells has the general shape of a diamond when in the expanded configuration.

Paragraph X: The stent of Paragraph W, wherein the second one of the cells is directly adjacent to each of the first one and the third one of the cells.

Paragraph Y: The stent of Paragraph M, further comprising a coronary section at least partially defined by the distal edge of the annulus section and the aortic section.

Paragraph Z: The stent of Paragraph Y, wherein the coronary section has fewer cells than each of the annulus section and the aortic section.

Paragraph AA: The stent of Paragraph M, wherein the distal edge of the annulus section extends continuously along a plurality of struts so as to span at least two cells located on a proximal side of the plurality of struts.

1. A stent for use with a prosthetic heart valve for replacement of a native valve, the stent comprising:
an expandable stent body having a collapsed configuration, an expanded configuration, an inflow end, an outflow end, and an annulus section at the inflow end;
at least one circumferential row of cells defined by struts of the stent body, the at least one row of cells including a first cell, a second cell, and a third cell, the second cell being positioned between the first and third cells;
wherein a distal edge of the annulus section is defined by a series of at least three struts, a first one of the struts defining a portion of the first cell, a second one of the struts defining a portion of the second cell, and a third one of the struts defining a portion of the third cell;
wherein each point on the second one of the struts is nearer, or equally near, the inflow end compared to each point on the first one of the struts and the third one of the struts.

2. The stent of claim 1, wherein the second cell is directly adjacent each of the first and third cells.

3. The stent of claim 1, wherein the stent body includes an aortic section at the outflow end, and a coronary section between the annulus section and the aortic section.

4. The stent of claim 3, wherein the distal edge of the annulus section includes a generally parabolic section that extends across at least part of a circumference of the stent body, the generally parabolic section having one proximal section that is relatively close to the inflow end and at least two distal sections that are more distant from the inflow end than the one proximal section.

5. The stent of claim 4, wherein the one proximal section of the generally parabolic section defines at least part of the second cell.

6. The stent of claim 5, wherein the annulus section includes at least one continuous circumferential row of cells, the aortic section includes at least one continuous circumferential row of cells, and the coronary section includes fewer cells than each of the annulus section and the aortic section.

7. The stent of claim 6, wherein the coronary section is at least partially defined by the at least one continuous circumferential row of cells in the aortic section and the distal edge of the annulus section.

8. The stent of claim 5, wherein the generally parabolic section of the annulus section is defined by at least five struts extending at least partially around the circumference of the stent body.

9. The stent of claim 4, further comprising a plurality of commissure attachment features, wherein one of the distal sections is connected to a first one of the commissure attachment features and the other of the distal sections is connected to a second one of the commissure attachment features.

10. A prosthetic heart valve, comprising the stent of claim 4 and a plurality of prosthetic valve leaflets mounted inside the stent body, wherein at least one of the prosthetic valve leaflets is attached to the stent body along a portion of the distal edge of the annulus section.

11. A stent for use with a prosthetic heart valve for replacement of a native valve, the stent comprising:
an expandable stent body having a collapsed configuration and an expanded configuration;
an annulus section at an inflow end of the stent body;
an aortic section at an outflow end of the stent body; and
a plurality of commissure attachment features positioned circumferentially around the stent body,
wherein a distal edge of the annulus section is generally parabolic and extends from one of the commissure attachment features to another of the commissure attachment features.

12. The stent of claim 11, wherein the distal edge of the annulus section is defined by a plurality of struts extending at least partially around the circumference of the stent body.

13. The stent of claim 11, wherein the distal edge of the annulus section is defined by at least three connecting struts extending at least partially around the circumference of the stent body.

14. The stent of claim 13, wherein the at least three connecting struts each define at least a portion of each of a plurality of adjacent cells in the annulus section.

15. The stent of claim 14, wherein, when the stent body is in the expanded configuration, a first one of the cells is generally diamond-shaped.

16. The stent of claim 15, wherein a second one of the cells includes a curved distal section and a curved proximal section.

17. The stent of claim 15, wherein a second one of the cells includes at least two substantially straight distal sections and at least two substantially straight proximal sections.

18. The stent of claim 17, wherein the at least two substantially straight distal sections are connected to the at least two substantially straight proximal sections by a vertical strut.

19. The stent of claim 15, wherein a second one of the cells includes at least two curved distal sections connected to at least two curved proximal sections by a vertical strut.

20. The stent of claim 15, wherein the distal edge of the annulus section has one proximal section that is relatively close to the inflow end and at least two distal sections that are more distant from the inflow end than the one proximal section, the one proximal section defining a portion of a second one of the cells.

21. The stent of claim 20, wherein a third one of the cells has the general shape of a diamond when in the expanded configuration.

22. The stent of claim 21, wherein the second one of the cells is directly adjacent to each of the first one and the third one of the cells.

23. The stent of claim 11, further comprising a coronary section at least partially defined by the distal edge of the annulus section and the aortic section.

24. The stent of claim 23, wherein the coronary section has fewer cells than each of the annulus section and the aortic section.

25. The stent of claim 11, wherein the distal edge of the annulus section extends continuously along a plurality of struts so as to span at least two cells located on a proximal side of the plurality of struts.
